(19) United States
(12) Patent Application Publication
(10) Pub. No.: US 2011/0190570 A1
(43) Pub. Date: Aug. 4, 2011

(54) BIOFEEDBACK INTERFACE DEVICE AND METHODS FOR CONTROLLING HEARTRATE IN RESPONSE TO SIGNALS

(76) Inventor: Michael Zaimi, Irvine, CA (US)

(21) Appl. No.: 12/914,819

(22) Filed: Oct. 28, 2010

Related U.S. Application Data
(60) Provisional application No. 61/337,284, filed on Feb. 1, 2010.

(51) Int. Cl. A61M 21/00

(52) U.S. Cl. 600/28

(57) ABSTRACT

Elevation in resting heart rate is associated with anxiety disorders and depression. Biofeedback techniques enable individuals to regulate involuntary physiological parameters such as heart rate. Embodiments of the invention described herein relate to a compact wearable biofeedback interface device which alerts the wearer when their measured heart rate exceeds a preselected individualized threshold. Once alerted of an elevation in heart rate the user can take steps to reduce their heart rate such as taking medication, employing meditative or relaxation techniques or seeking medical treatment.

### RELATED APPLICATIONS

This application claims priority under 35 U.S.C. §119(e) to U.S. Provisional Application No. 61/337,284 filed Feb. 1, 2010, which is hereby incorporated by reference in its entirety.

### FIELD OF THE INVENTION

The present embodiments relate to a biofeedback interface device which alerts the user when measured heart rate (beats per minute) exceeds a user defined threshold and methods for using said biofeedback interface device to prompt the user to employ relaxation and meditation techniques to return the measured heart rate within determined limits. Some embodiments relate to the treatment of depression. Some embodiments relate to the treatment of anxiety disorders.

### BACKGROUND OF THE INVENTION

Heart rate is expressed as the number of heart beats per unit of time, typically beats per minute (BPM). An individual's resting heart rate is typically between 60-80 BPM, but may be higher or lower than average depending upon an individual's age, gender, and level of physical fitness. For example a middle-aged, sedentary individual may have a resting heart rate which exceeds 100 BPM, while many elite athletes have a resting heart rate in the range of 28-40 BPM. Some studies have indicated that a high resting heart rate may be a risk factor for cardiovascular disease, possibly due to increased stress on the heart muscle.

Heart rate adapts to changes in the body's need for oxygen. When an individual is active, such as during periods of exercise, heart rate increases. When an individual is relaxed and at rest, heart rate decreases. Heart rate is also affected by stress. The body reacts to stressful stimuli by releasing adrenaline and cortisol, which increases heart rate, redirects blood flow to the muscular system, releases fats into the bloodstream for use as energy, increases breathing rate, tenses muscles, and increases your blood's clotting ability. While these reactions may be beneficial in a fight-or-flight situation, prolonged periods of stress negatively affects health.

Biofeedback techniques represent an effective way to self-regulate heart rate. Biofeedback is a process that enables an individual to learn how to influence involuntary physiological functions for the purposes of improving health and performance. Biofeedback techniques have been widely used to treat: Migraine and tension headaches, digestive disorders, such as irritable bowel syndrome, hypertension, hypotension, cardiac arrhythmias, Raynaud's, epilepsy, paralysis, movement disorders, and chronic pain.

Traditionally, biofeedback has employed computers on which patient are trained by health professionals. Biofeedback machines detect a patient's physiological functions with a high degree of sensitivity and translate the information into stimuli that the patient can detect, such as activating a light, changing patterns on a computer monitor or activating a buzzer. The patient then “practices” manipulating physiological functions and the biofeedback machine allows the patient to track their success. The ability to control the feedback signals may act as a reward, further reducing tension. Wide adoption of biofeedback as an effective method to self-regulate response to stressful stimuli has been hampered by limited availability of biofeedback machines and western reliance on medications to treat physiological disturbances.

### SUMMARY OF THE INVENTION

An embodiment relating to a method for promoting self-regulation of heart rate comprising: providing a subject with a compact, wearable biofeedback interface device that measures heart rate and alerts the subject when the subject's heart rate exceeds a selected threshold, and instructing said subject to respond to a signal generated by the device by employing relaxation and meditative techniques, wherein said subject continues said techniques until the subject's heart rate drops below the threshold.

An embodiment relating to a method for promoting self-regulation of heart rate comprising: providing a subject with a compact, wearable biofeedback interface device that measures heart rate and alerts the subject when the subject's heart rate exceeds a selected threshold, and instructing said subject to respond to a signal generated by the device by employing relaxation and meditative techniques, wherein said subject continues said techniques until the subject's heart rate is reduced.

An embodiment relates to a method for promoting self-regulation of heart rate comprising: providing a subject with a compact, wearable biofeedback interface device comprising a pressure sensor which measures the subject's heart rate by converting mechanical force generated by the subject's pulse to a voltage signal, wherein an electronic circuit conditions said signal, processes said signal, compares said processed signal to a threshold and when said signal exceeds said threshold, activates a signaling device alerting the subject; and instructing said subject to respond to a signal generated by the device by employing relaxation and meditative techniques, wherein said subject continues said techniques until the subject's heart rate drops below the threshold.

An embodiment relating to a method of preventing or reducing the frequency of episodes of anxiety comprising: wearing a compact wrist-mounted biofeedback device; receiving a signal from the biofeedback device when measured heart rate exceeds a predetermined threshold; and responding to said signal by engaging in meditative techniques.

An embodiment relating to a method of preventing or reducing the frequency of episodes of anxiety comprising: wearing a compact wrist-mounted biofeedback device comprising an adjustable wristband provided on the wrist-adjacent surface with a force sensing resistor that changes in input voltage proportional to force applied by pulsations of a radial artery connected to an electrical circuit comprised of a gain and bias stage, a low-pass filter stage, a modulation stage, an LED, a frequency to voltage converter linked to an alarm; receiving a signal from the biofeedback device when measured heart rate exceeds a predetermined threshold; and responding to said signal by engaging in meditative techniques.

An embodiment relating to a method of preventing or reducing the frequency of episodes of depression comprising: wearing a compact wrist-mounted biofeedback device; receiving a signal from the biofeedback device when measured heart rate exceeds a predetermined threshold; and responding to said signal by engaging in meditative techniques.

An embodiment relating to a method of preventing or reducing the frequency of episodes of depression comprising: wearing a compact wrist-mounted biofeedback device comprising an adjustable wristband provided on the wrist-adjacent surface with a force sensing resistor that changes in input voltage proportional to force applied by pulsations of a radial artery connected to an electrical circuit comprised of a gain and bias stage, a low-pass filter stage, a modulation stage, an LED, a frequency to voltage converter linked to an alarm; receiving a signal from the biofeedback device when measured heart rate exceeds a predetermined threshold; and responding to said signal by engaging in meditative techniques.

A kit for promoting self-regulation of heart rate in a subject is disclosed in accordance with another embodiment of the invention. The kit comprises: a) a compact, wearable biofeedback interface device that signals the wearer when the wearer's heart rate exceeds a pre-determined threshold; b) instructions that teach the subject to respond to the biofeedback stimulus by practicing a relaxation and/or meditative technique until the subject's heart rate drops below the threshold; and c) an aid to relaxation and/or meditation. In some embodiments, the wearable biofeedback device may comprise a pressure sensor which measures the subject's heart rate by converting mechanical force generated by the subject's pulse to a voltage signal, an electronic circuit that compares the signal to a pre-selected threshold so that when the signal exceeds the threshold, a biofeedback stimulus that can be perceived by the subject is activated. In some embodiments, the aid may be an instructional CD or DVD that teaches the relaxation and/or meditative technique. Alternatively, the aid may be a memory device comprising digitalized music and/or video.

### BRIEF DESCRIPTION OF THE DRAWINGS

FIG. 1 shows an embodiment of a biofeedback interface device. The numeral 1 shows a force sensing resistor; numeral 2 shows a thumbwheel variable resistor, numeral 3 shows an LED light, numeral 4 shows a back plate, numeral 5 shows a buzzer alarm, numeral 6 shows a wrist strap.

FIG. 2 shows an embodiment of a Force Sensing Resistor. The numeral 7 shows an active area with printed interdigitating electrodes. The numeral 8 shows a flexible substrate with printed semi-conductor.

FIG. 3 shows an electrical circuit block-diagram.

FIG. 4 shows a FSR Voltage Divider Configuration and a Family of Force vs. V_{OUT} Curves.

FIG. 5 shows a FSR Gain and Bias Stage. In this embodiment, an operating point, adjustable gain stage and adjustable bias are combined.

FIG. 6 shows the Sallen-Key topology for a second order Butterworth (no ripple) low-pass filter (LPF). In this embodiment, the cut-off frequency for a −3 dB decay has been set to 20 Hz based on the response of the human pulse collected off the surface of the wrist skin. An overall gain of 1 was also applied through this stage. Based on these specifications, the different parameters of RA, RB, CA and CB were calculated and displayed in FIG. 6.

FIG. 7 shows a Bode Diagram of a Low-Pass Filter.

FIG. 8 shows a Frequency Detector which functions without the use of a microprocessor.

FIG. 9 shows the curves obtained by using a simulated differential signal and applying an overall +2.5 V bias to obtain only positive voltages such as the ones collected for the wrist pulse. Every time that the harmonic filter output signal (dashed line) decreases with respect to the envelope signal (solid horizontal line), a digital pulse is generated (square wave). This square wave varying with the frequency of the pulse can be used to illuminate an LED such as the SLR322-DC. This way the patient can make sure that the device is properly positioned and can also see his/her pulse.

FIG. 10 shows the schematics of an embodiment of a frequency-to-voltage converter IC. In this embodiment, the pulses at different frequencies enter the converter from the bottom left pin and are converted to a voltage Vₒᵤₜ as shown on the right hand side plot. +Vₒᵤₜ constitutes one side of the comparator. In some embodiments, the other side can be built using a voltage divider circuit with a variable resistor that can be set by the user. By changing this variable resistor, the user can set the threshold voltage reflecting the maximum heart-rate allowed for that patient. This signal constitutes the other side of the comparator. These two signals are then compared and the output drives the NPN transistor, which in turn drives the actuator.

FIG. 11 shows one embodiment of a biofeedback interface device.

FIG. 12 shows the Pulse Signal compare to the Gain and Bias Signal over time.

FIG. 13 shows the Filtered Signal compared to the Gain and Bias Signal.

FIG. 14 shows the Frequency Detector vs. Modulation Inputs.

FIG. 15 shows the Alarm vs. Frequency signal.

### DETAILED DESCRIPTION OF THE PREFERRED EMBODIMENTS

Increase in heart rate can be used as an indicator of the level of physiological and emotional distress experienced by an individual. Embodiments of a biofeedback interface device described herein provides the user with a warning that the user's heart rate has exceeded a set threshold, which allows the user to employ conscious techniques to reduce stress and bring heart rate within set levels.

Anxiety Disorders are the most common mental illness in the U.S. with 19.1 million (13.3%) of the adult U.S. population (ages 18-54) affected. According to “The Economic Burden of Anxiety Disorders,” a study commissioned by the ADAA and based on data gathered by the association and published in the Journal of Clinical Psychiatry, anxiety disorders cost the U.S. more than $42 billion a year, almost one third of the $148 billion total mental health bill for the U.S. More than $22.84 billion of those costs are associated with the repeated use of healthcare services, as those with anxiety disorders seek relief for symptoms that mimic physical illnesses. People with an anxiety disorder are three-to-five times more likely to go to the doctor and six times more likely to be hospitalized for psychiatric disorders than non-sufferers. Traditionally anxiety disorders have been treated with medications, which are costly and often have adverse side effects.

One severe form of anxiety disorder is panic disorder, commonly termed, “panic attacks.” Suffers of panic attacks, report a sudden onset of fear and apprehension characterized by heart palpitations, shortness of breath, sweating, and dizziness. These physical symptoms cause discomfort and alarm, which leads to increased anxiety, and forms a positive feedback loop. Panic attack suffers often report avoiding social situations or even leaving their house for fear that a panic attack may occur without warning.

Biofeedback techniques have been shown to be effective in treating anxiety disorders. One physiological parameter that can be used to monitor the onset of an episode of anxiety is an increase in resting heart rate. Embodiments of the present invention relate to a biofeedback interface device that measures a user's heart rate and alerts the user when the measured heart rate exceeds a preselected threshold. Such threshold is personalized, as an individual's resting heat rate is influenced by factors such as age, fitness, and gender. The means for alerting the subject may be any means known in the art for generating a biofeedback stimulus which is capable of being perceived by the subject, including e.g., a audible alarm, a visual alarm, a tactile alarm (e.g., vibration), and combinations thereof. Once alerted to their elevated heart rate, the user can employ relaxation and meditative techniques to bring their heart rate below the threshold level. In this way, the user can identify an episode of anxiety early, before symptoms manifest themselves and form a feedback loop. Additionally users of said biofeedback monitoring device can be taught to recognize and avoid situations that commonly lead to episodes of anxiety.

Embodiments of the biofeedback interface device described herein are also useful in treating individuals with anger-control issues. When an individual begins to feel angry, their heart rate goes up. An individual wearing an embodiment of a biofeedback interface device described herein is alerted to their increasing agitation, and can remove themselves from the situation before it progresses.

The World Health Organization has found that Major Depression was the leading cause of disability worldwide. Depression causes suffering, decreases quality of life, and impairs social and occupational functioning. It is associated with increased health care costs as well as with higher rates of many chronic medical conditions. Studies have shown that a high number of depressive symptoms are associated with poor health and impaired functioning, whether or not the criteria for a diagnosis of major depression are met.

Depression is characterized by changes in mood, self-attitude, cognitive functioning, sleep, appetite, and energy level. Depression is also associated with elevated heart rate and reduced heart rate variability (HRV). HRV is a physiological phenomenon where the time interval between heart beats varies. Reduced HRV has been shown to be a predictor of mortality after myocardial infarction.

The elevated heart rate associated with depression may be treated by providing suffers of depression with biofeedback interface devices according to the present embodiments. Feedback of physiological conditions, such as elevated heat rate, provides information to help patients recognize a depressed state. Once the depressed state is recognized, the user of the biofeedback interface device may employ meditative techniques or consciously supplant negative thoughts with more positive thoughts in order to bring their heart rate under a set threshold. The ability to control a physiological condition such as heart rate may act as a reward, providing the user with a sense of control, which further reduces tension and elevates mood. The methods described herein may optionally be practiced in combination with medication and psychotherapy.

Several embodiments disclosed herein relate to a device which measures heart rate off the human wrist. Some embodiments of the device compare the measured heart rate to a user-selected heart rate threshold. Some embodiments of the device compare the measured heart rate to a physician-selected heart rate threshold. In several embodiments, when the measured heart rate exceeds the selected heart rate threshold, a perceptible signal is transmitted to the user. In some embodiments the signal is terminated after transmission. In other embodiments, the signal continues until the user's heart rate falls below the selected threshold. In preferred embodiments, when the measured heart rate exceeds the user-selected heart rate threshold, an internal vibrator is activated. Perception of the signal transmitted by the device alerts the user that their heart rate has exceeded the pre-set limit, allowing the user to take action. The user can respond to the signal by taking medication, practicing breathing techniques, meditating, changing their line of thought or activity or by seeking medical assistance.

The threshold heart rate may be set to about 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, or 195.

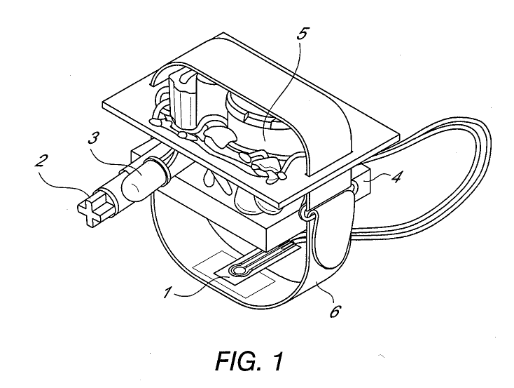

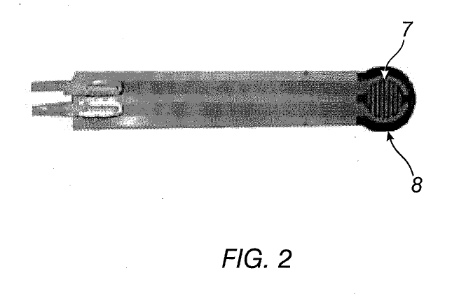

Certain embodiments relate to a biofeedback interface comprising a pressure sensor in contact with the surface of the skin, which measures the patient's heart rate and conveys that information to the electronics of the device. In some embodiments, the pressure sensor is comprised of an active area with printed interdigitating electrodes as shown in FIG. 2, numeral 7, and a flexible substrate with printed semi-conductor as shown in FIG. 2, numeral 8. In several embodiments the biofeedback interface comprises an electrical circuit which receives the mechanical information from the human pulse and performs signal conditioning, signal processing, comparison of the processed signal to a heart-beat threshold set by the patient, and drives a vibrator so as to warn the user when his/her heart-beat has surpassed the selected threshold. In some embodiments a back plate or insulating material is positioned on the wrist-facing surface of the circuit board. An example of a back plate is shown at FIG. 1, numeral 4. Several embodiments incorporate a strap or bracelet to position the biofeedback interface on a wrist. Certain embodiments incorporate a Velcro bracelet. In certain embodiments, the biofeedback interface resembles a wristwatch. In several embodiments, a wireless signal is transmitted when the wearer's heart rate exceeds a pre-determined threshold. In some embodiments the user interface transmits a detectable signal to the user, alerting the user when the measured heart rate exceeds a predefined threshold. In some embodiments a signal is transmitted to the user's physician or a monitoring center, alerting the user's physician or monitoring center when the measured heart rate exceeds a predefined threshold.

In some embodiments a back plate or insulating material is positioned on the wrist-facing surface of the circuit board. An example of a back plate is shown at FIG. 1, numeral 4. Several embodiments incorporate a strap or bracelet to position the biofeedback interface on a wrist. Certain embodiments incorporate a Velcro bracelet. In certain embodiments, the biofeedback interface resembles a wristwatch.

Some embodiments relate to a biofeedback interface comprising a sensor that measures the electrical signals that are generated by the beating heart and transmits the electrical signal to a user interface. In some embodiments the sensor is positioned on the user's chest in proximity with the user's heart. In several embodiments, transmission is wireless. In some embodiments the user interface transmits a detectable signal to the user, alerting the user when the measured heart rate exceeds a predefined threshold. In some embodiments a signal is transmitted to the user's physician or a monitoring center, alerting the user's physician or monitoring center when the measured heart rate exceeds a predefined threshold.

In some embodiments, the wristband, strap or bracelet has low-elastic properties so as to reduce elongation during heart rate measurements. Low cost, durability, ease-of adjustment, appearance and practicality are other qualities to be considered in choosing a suitable material for the wristband, strap or bracelet. In some embodiments the wristband comprises a Velcro bracelet attached to a plastic loop at one of its extremities that allows adjustment to any wrist size for both adults and children. In some embodiments, the wristband, strap or bracelet is customizable with different colors or design motifs.

In several embodiments, the biofeedback interface comprises a sensor for detecting heart rate. In some embodiments the heart rate is detected with a pressure sensor. In some embodiments, a strain sensor placed on or near a pulse point is used to measure heart rate. Points for measuring the heart rate include: the ventral aspect of the wrist on the side of the thumb (radial artery); the ulnar artery; the neck (carotid artery); the inside of the elbow, or under the biceps muscle (brachial artery); the groin (femoral artery); behind the medial malleolus on the feet (posterior tibial artery); middle of dorsum of the foot (dorsalis pedis); behind the knee (popliteal artery); over the abdomen (abdominal aorta); the chest (apex of heart). In an embodiment, the sensor is a Force Sensing Resistor (FSR). A FSR sensor changes in resistance given an input voltage proportional to the pressure on the sensor when a force is applied against its active area. An example of a FSR is shown in FIG. 2.

In an embodiment, the FSR is placed on the wrist so that it is in close proximity with the radial artery, which provides measurable pulsating force. When the heart beats, the radial artery will pulse and cause a deflection in the sensor that will send a signal. The radial artery force due to pulses applies a stimulus to the FSR. In one embodiment, one side of the sensor is in close contact with the surface of the wrist skin and the other is in contact with the wristband, strap or bracelet holding it in place. In some embodiments, a semi-hard surface is provided on at least on one of the two surfaces in contact with the sensor in order to improve signal measurement. In some embodiments, a semi-hard yet absorbing material is used so as to minimize the vibrations caused by involuntary wrist movements. In an embodiment, a similar material to the wristband is used to stabilize the axis of the sensor mechanically and to connect the sensor to the bracelet, creating the biofeedback interface. In some embodiments, an LED light blinks in time with the users pulse, notifying them that the biofeedback device is properly positioned.

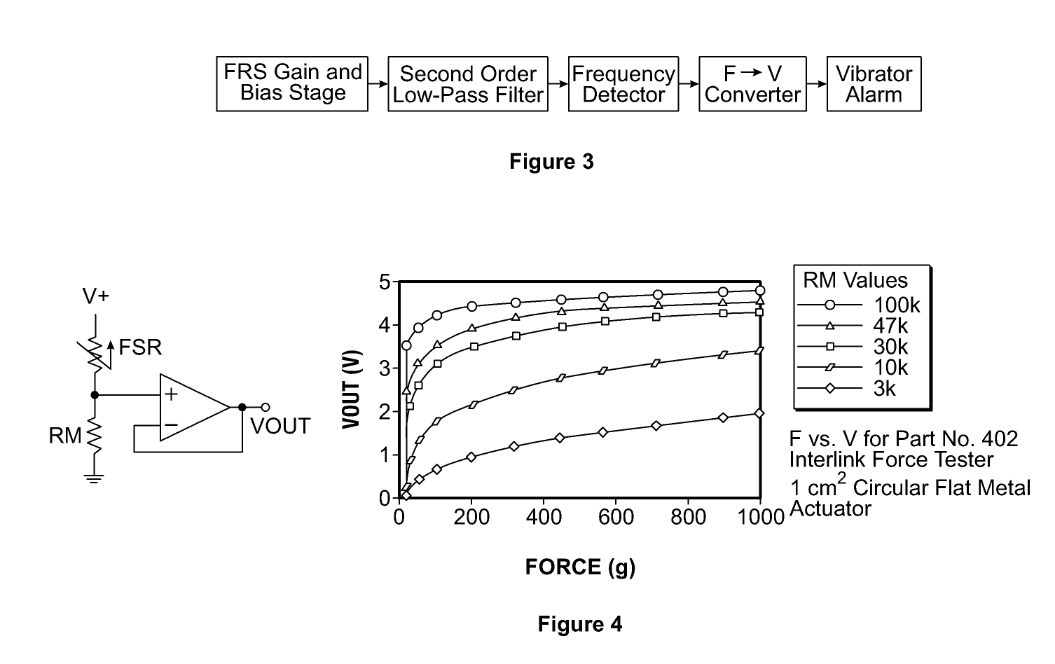

In several embodiments, the biofeedback interface comprises an electrical circuit comprising the following stages: a FSR gain and bias stage; a second order low-pass filter; a frequency detector; a frequency to voltage converter; and a signaling device. In some embodiments, the signaling device is a vibrator. Other examples of a signaling device include, but are not limited to, a digital display, a light, or an audible alarm. More than one signaling device may be optionally included. An embodiment of an electrical circuit is depicted in FIG. 3.

In several embodiments, the FSR device is tied to a measuring resistor in a voltage divider configuration for simple force-to-voltage conversion. The output of the FSR may be described by the equation: V_{OUT}=(V+)/[1+RFSR/RM]. An example of the FSR voltage divider configuration is shown at FIG. 4. In the embodiment of FIG. 4, the output voltage increases with increasing force. In one embodiment, the measuring resistor, RM, is chosen to maximize the desired force sensitivity range and to limit current. In an embodiment, the current through the FSR is limited to less than 1 mA/square cm of applied force. Suggested opamps for single sided supply designs are LM358 and LM324. In some embodiments, a quad opamp LM324 is used. In other embodiments, LM358 IC is used to minimize the size of the electrical circuit. In some embodiments, FET input devices such as LF355 and TL082 are used. The low bias currents of these op-amps reduce the error due to the source impedance of the voltage divider. FIG. 4 shows a family of FORCE vs. V_{OUT} curves for a standard FSR in a voltage divider configuration with various RM resistors.

The mechanical forces generated by the pumping of the heart are quite small. Therefore a large voltage change is desired for a small change in the force imposed on the sensor. In one embodiment, an empirical value of RM=100 kOhm was found to be suitable for the FSR-400. One of ordinary skill in the art would understand that smaller RM values would make the signal “lazier” whereas larger RM values make the sensed signal almost instantaneous and thus would adjust the RM values to achieve the desired signal. In the embodiment of FIG. 4, once RM is set to a fixed optimized value, the operating point of the FSR is set.

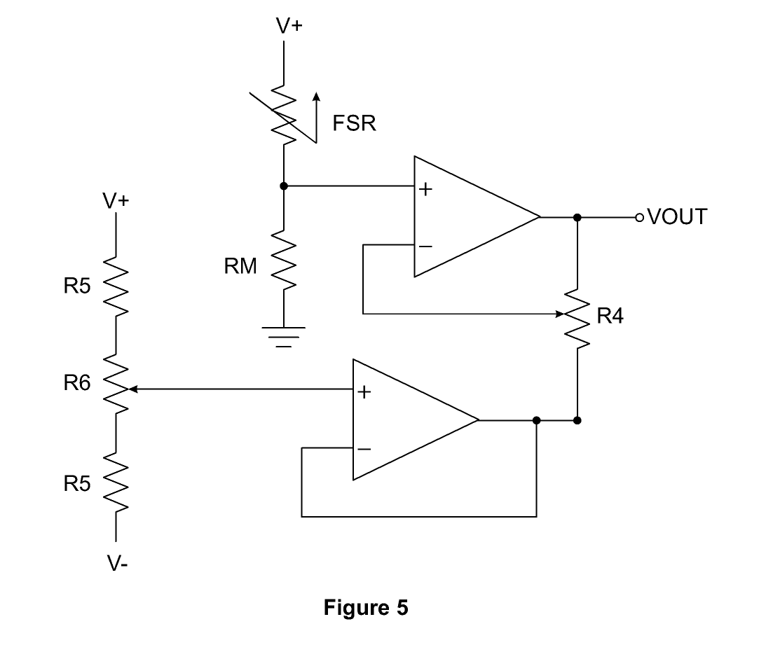

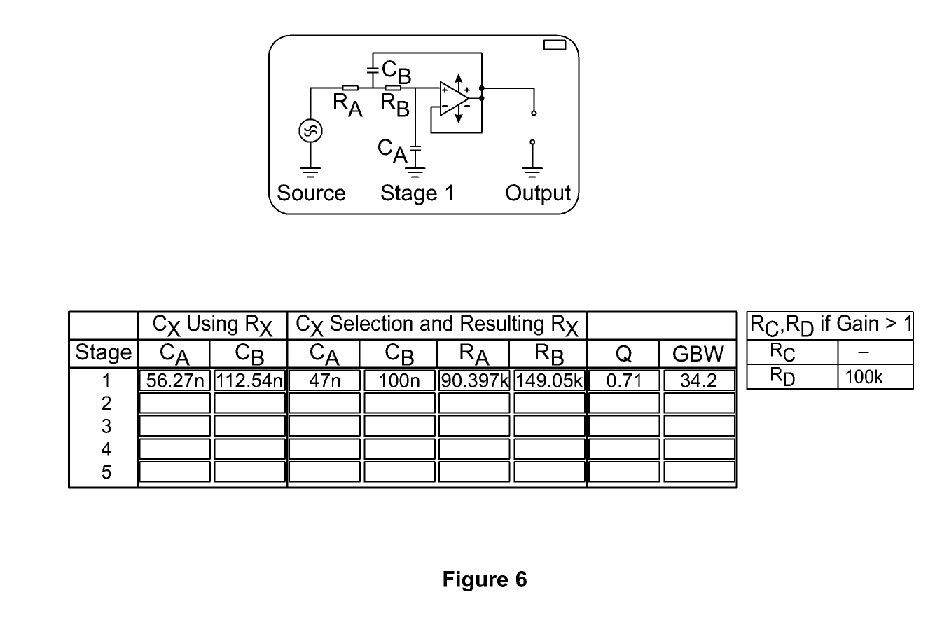

Because the sensed signals are in the order of few 100 mV, in one embodiment, an amplification stage is used to increase the amplitude of the collected pulses. This embodiment results in an improvement to the signal-to-noise ratio of the signal and the signal conditioning and processing stages further down the electrical circuit. In one embodiment, as shown in FIG. 5, an operating point, adjustable gain stage, and an adjustable bias are combined using a buffer circuit. The left part of the diagram of FIG. 5 shows the adjustable bias. The presence of the adjustable bias stops the feedback current through pot R4 to lower the voltage on the output pin of the lower opamp. A minimum bias may be obtained by adjusting R6 (set to 10 kOhm) to its lowest position. Adjustable gain can be obtained by varying the value of resistance R4. For example, by setting R4=100 kOhm and setting its position to the highest one on this diagram, a maximum gain is obtained with a fast signal response without saturation and without losing the signal characteristics.

Similar to the FSR voltage divider introduced in the embodiment of FIG. 4, the interface embodiment of FIG. 5 isolates the output from the high source impedance of the FSR. The ratio of R4 may be adjusted to set the gain of the output. In this manner, a broad range gain adjustment can be made. In addition, a circuit of this embodiment allows the isolation of the offset trim from the adjustable gain. With this separation, there is no constraint on values for the pot. Typical values for R5 and the pot R4 are around 10 kOhm. In an embodiment, both (−) and (+) power supplies are required, which means using two batteries instead of one. A non-limiting example of a suitable battery is a CR1220 (3 Volts) battery, which is very light and very thin. Other batteries may also be used.

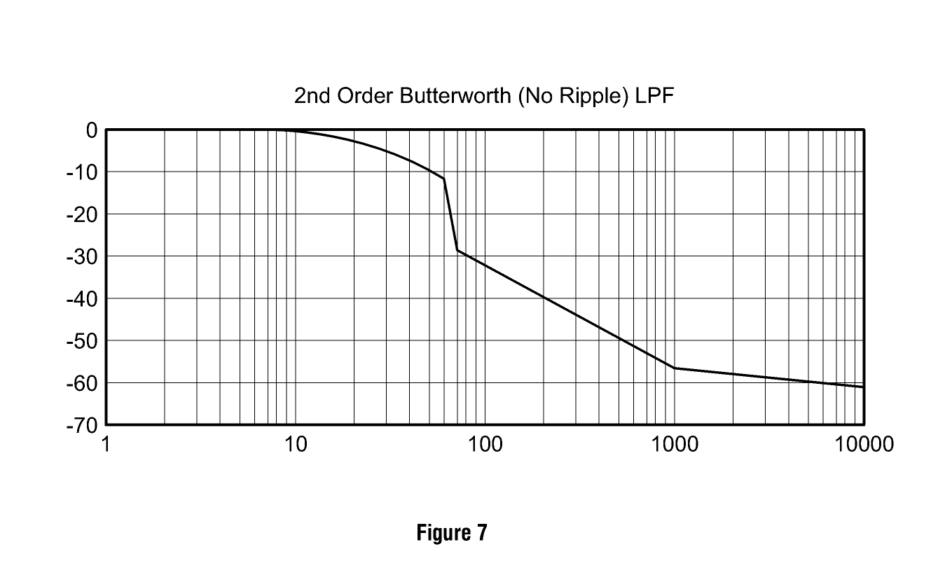

In several embodiments, regular ceramic resistors and capacitors can be used for the prototyping phase and surface-mount elements for the final PCB. In one embodiment, a TL072 8-pin IC is used to emulate the opamp due to its low-noise characteristics. The Bode diagram (gain only as phase shift does not matter in this application) of this FLP is shown in FIG. 7. In this embodiment, LPF has been tuned for a cut-off frequency of 20 Hz in order to fit the heart-rate application. The plot clearly shows a drop of −3 dB at 20 Hz as expected. A second order filter is sufficient to filter out the skin vibrations and unwanted wrist movements that may introduce an error to the pulse measurement. In some embodiments, a frequency detector can be used to detect the peaks of the signal generated by the pulse at the wrist.

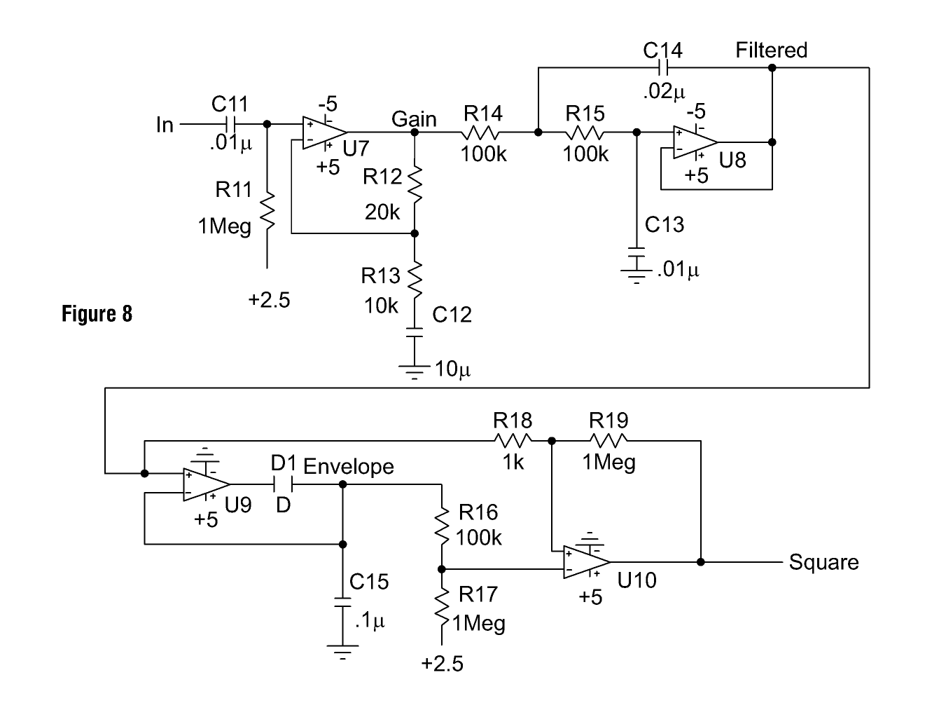

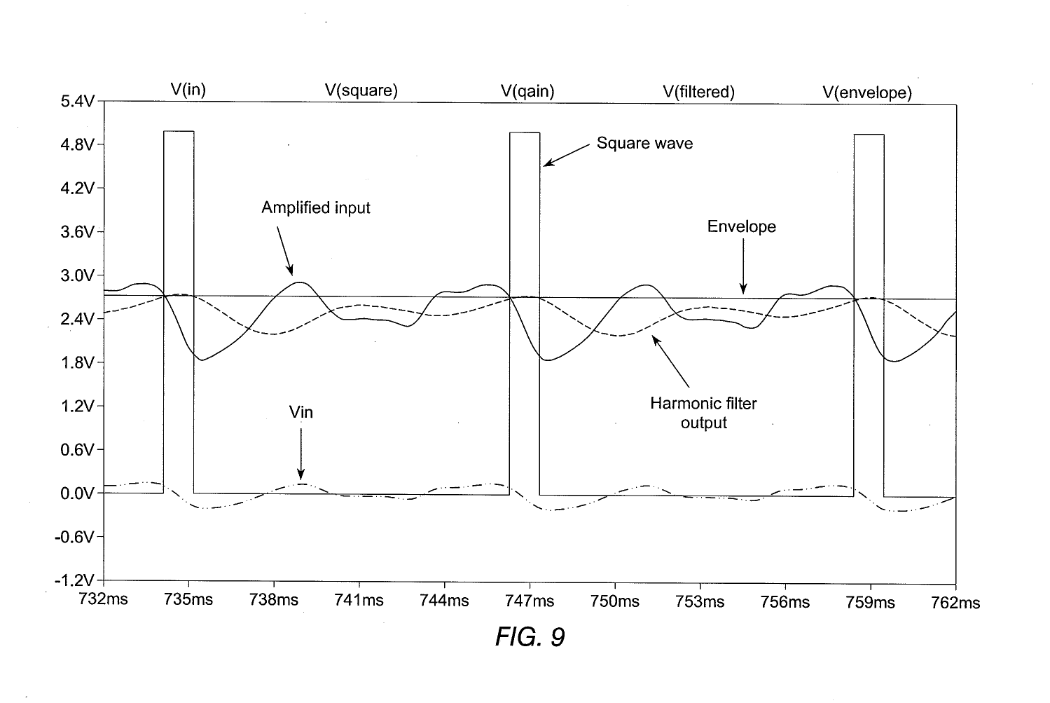

In several embodiments, a frequency detector circuit, which generates a positive peak every time that the measured signal reaches a maximum, is used to generate a digital ON output at every peak of the measured pulse. The embodiment of FIG. 8 accomplishes this task in two stages. This embodiment includes the gain and bias stages and the LPF circuit. In this embodiment, the first stage is peak detection where capacitor C15 is charged so as to maintain the maximum voltage of the filtered signal. This signal has now become an envelope which is in turn compared to the filtered signal. Every time that the filtered signal decreases with respect to the envelope, the output of the comparator becomes positive and therefore generating a pulse at that frequency. For this reason, the circuit is called a frequency detector. The embodiment of FIG. 8 functions without the use of a microprocessor, resulting in reduced cost and complexity. In alternative embodiments, a microprocessor may be incorporated.

The values used for the different capacitors and resistors are not final and can be optimized using a simulator such as P-Spice before implementing them on the proto-board. In several embodiments, TL072 can be used as opamps and D4148 can be used as charging diode.

Once a digital output has been obtained every time that a pulse-like signal reaches its maximum, it converted back to voltage in order to compare it to the user-defined threshold. In several embodiments, the user-defined threshold is set by using a thumbwheel variable resistor. An example of a thumbwheel variable resistor is shown at FIG. 1, numeral 2. By rotating this variable resistor, the user sets the voltage divider across to a certain voltage. In one embodiment, the corresponding beats per minute (BPM) are printed on the rotary resistor. The voltage corresponding to the threshold heart rate (BPM) is then compared to the output of the circuit.

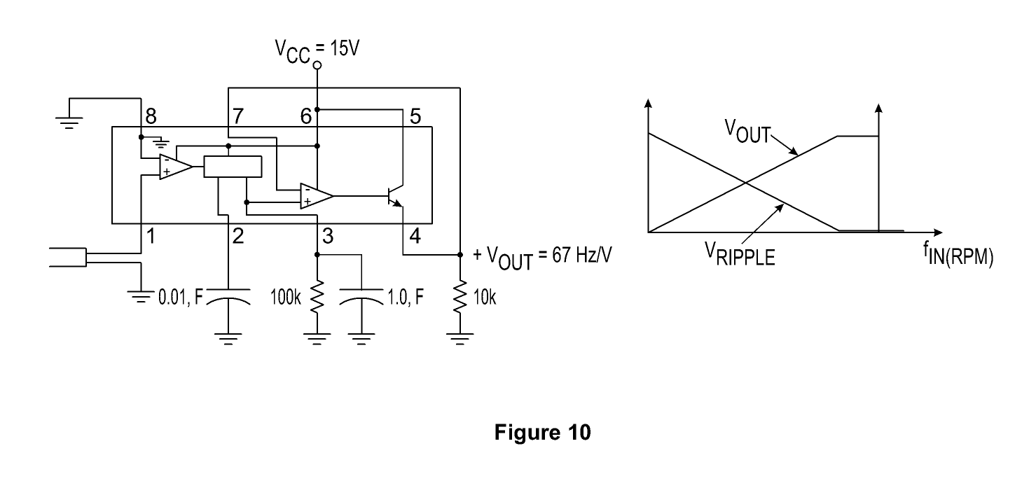

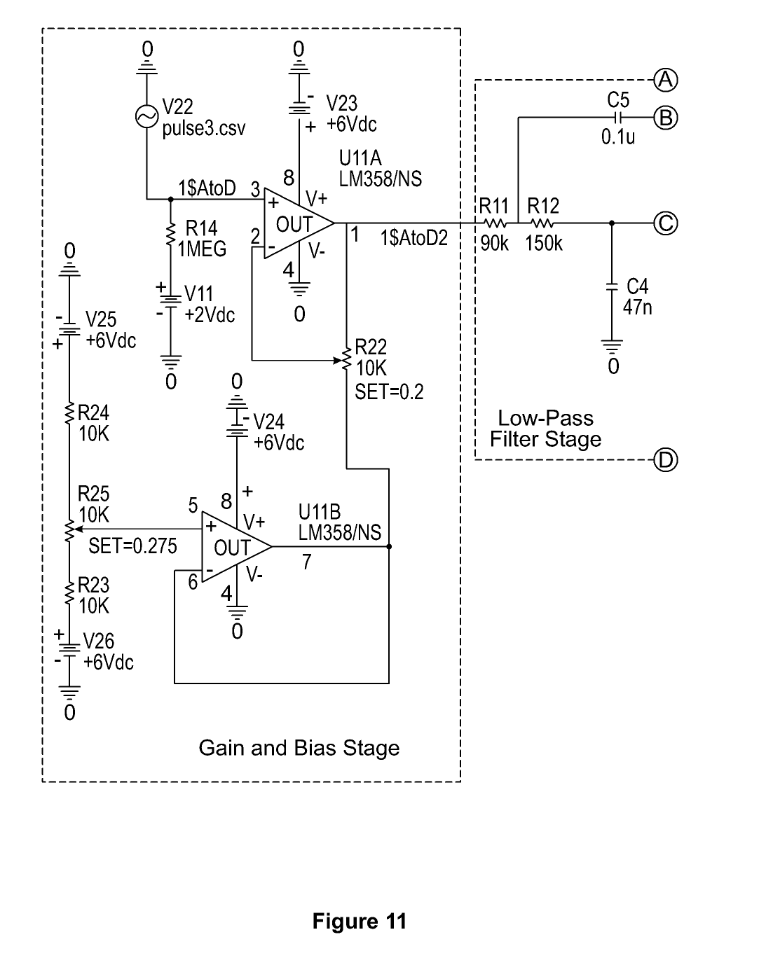

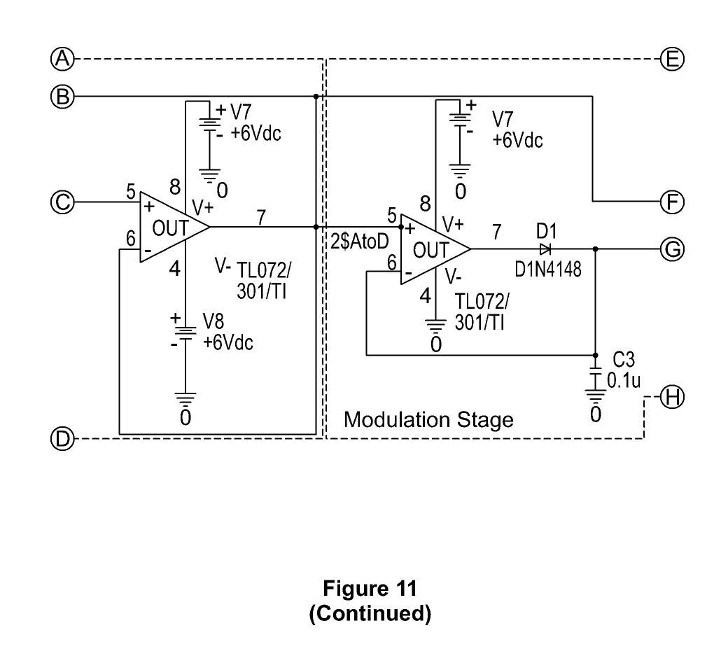

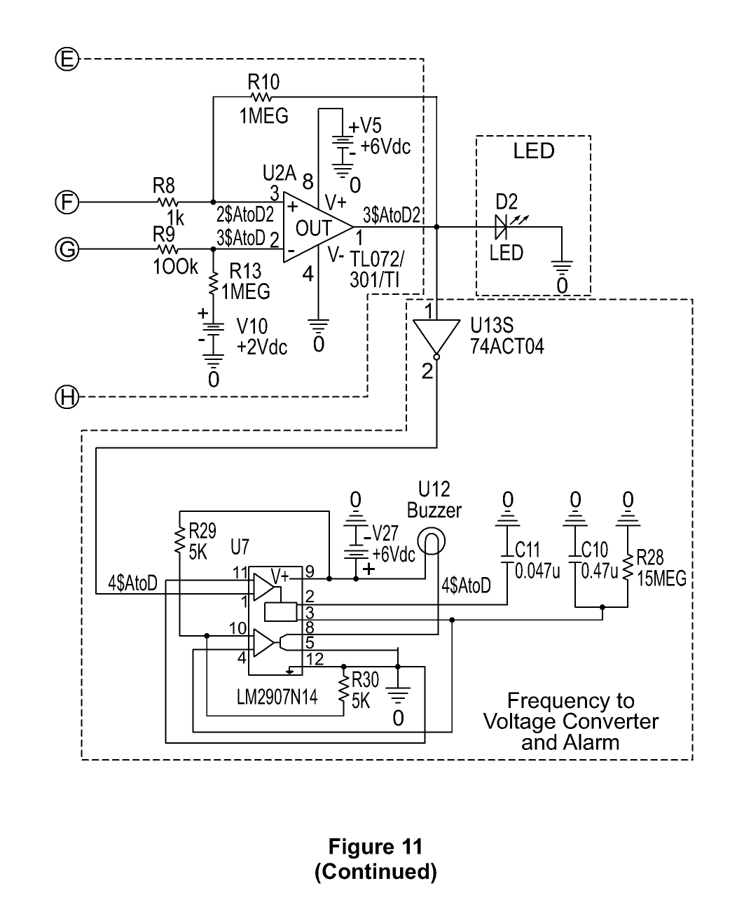

One embodiment of an integrated circuit (IC) including all of the functions described herein is LM2907 and LM2917 (with charging Zener diode). This 8-PIN IC converts a digital signal with variable frequency to a corresponding voltage output. This voltage can then be mapped to the heart rate in BPM. Since both threshold and converted signals are in Volts, they can be compared using a comparator. In some embodiments, the output of the comparator can feed the base of an NPN transistor and therefore, can drive a load such as a buzzer, a vibrator or a piezzoresistive beeper. An embodiment of an integrated circuit capable of converting a digital signal with variable frequency to a corresponding voltage output is shown in FIG. 10.

Some embodiments incorporate a vibrating alarm which alerts the user when their BPM exceeds the threshold. In some embodiments, the vibrator is a linear vibrator. Vibration mechanisms have the advantage of being discrete and consuming little power; however other devices may be used to signal the user. In some embodiments, the vibration mechanism will operate as long as the heart rate is surpassing the threshold. Other signaling devices may be used either in addition to the vibrating mechanism or as an alternative. Examples include audible alarms, such as a buzzer alarm, lights, and visual displays. Other examples include tactile alarms, such as activation of a mild heat source. In some embodiments, a digital display can be incorporated to show the measured heart-rate of the user.

In some embodiments, the biofeedback interface is comprised of passive elements set to fixed values. In some embodiments the threshold heart rate value may be automatically adjusted based on the user's activity level. In other embodiments, a microprocessor may be incorporated. In some embodiments, a stretch sensor may be used to create an adjustable feedback that subtracts the skin movement from the actual measured pulse. Some embodiments incorporate an automatic gain and bias control which sets the measured signal between a lower-voltage and upper-voltage levels. In some embodiments, a memory chip is incorporated so that so that the user's heart rate can be stored and later downloaded, which enables doctors to see the changes over a day or two days or even a week period. In some embodiments, the biofeedback interface may optionally include sensors to measure skin temperature and/or conductivity.

In several embodiments, the biofeedback interface may optionally incorporate a clock, timer, visual display, back-ground light, wake-up alarm, countdown, solar panel, allocated memory for recording heart-rate with possibility to recall previously-recorded heart-rates, external connection to computer for doctors and patients to analyze recorded data, and Bluetooth enabled to transfer data to receptive device such as cell phone, car, or similar devices.

In some embodiments, the biofeedback interface provides a visual representation of the user's heart rate. The visual representation may be in the form of a flashing LED light or a pulsating symbol on a visual display. In some embodiments the user is instructed to focus on the visual representation of the user's heart rate and to mentally attempt to control the visual stimulus.

Some embodiments relate to a kit comprising a biofeedback interface device capable of alerting a user when the measured BPM exceed a user defined threshold; and one or more of the following: a pamphlet providing information regarding stress avoidance and techniques for promoting positive thoughts and inducing relaxation; an audio CD providing instructions on relaxation and meditation techniques; a computer program or questionnaire which enables the user to identify stressful stimuli. Techniques for inducing relaxation include progressive relaxation, yoga, autogenic training, transcendental meditation, selecting a quiet environment, listening to soothing music, visualizing soothing scenes, and assuming a passive attitude.

Several embodiments relate to a method for treating depression or an anxiety disorder by providing the user of a biofeedback device according to the present embodiments with a signal alerting them to an elevated heart rate. Upon receiving a signal notifying them that their heat rate is elevated above a threshold level, the user can employ meditative and relaxation techniques to reduce their heart rate. Such methods can lead to a reduced reliance on medications and reduced long term health care costs as user are able to avoid the damage to their heart muscle caused by prolonged periods of stress.

The following Examples are presented for the purposes of illustration and should not be construed as limitations.

### Example 1

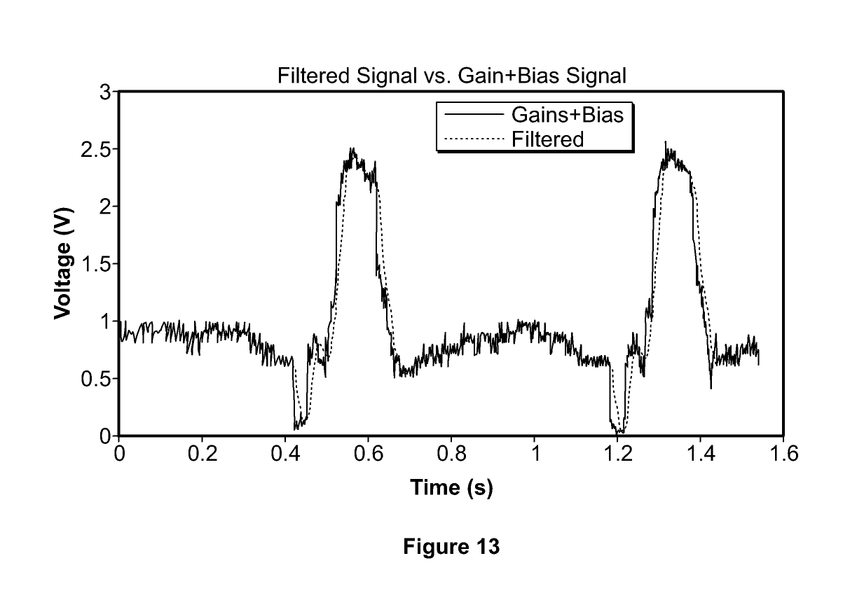

A prototype biofeedback interface device was constructed of a Velcro wrist strap having a plastic loop on one end through which the opposite end of the Velcro strap passes to form an adjustable loop with a sensor was mounted to the wrist-facing surface of the strap, as shown in FIG. 1, numeral 6. The sensor was comprised of: a force sensing resistor, as shown in FIG. 1, numeral 1, two three volt batteries, a second order filtering stage with a cutoff frequency of 20 Hz, an amplitude modulator, a comparator between threshold and actual sensed heart rate off the wrist, and a pulse generator with a LED, as shown in FIG. 1, numeral 3, connected to the pulse generator, a Frequency-to-Voltage converter, a voltage divider circuit, which created a threshold, a NPN transistor serving as a driver in case threshold is surpassed and a buzzer, as shown in FIG. 1, numeral 5, that alarms the user when measured heart rate exceeds the threshold. FIG. 13 shows a schematic of the assembly. The circuitry of the biofeedback interface device was insulated from contact with the skin of a user by a back plate, as shown in FIG. 1, numeral 4.

### Example 2

A subject (PM) having regular episodes of depression over 25 times in a two week period was provided with an embodiment of the invention as described in Example 1 and was instructed to wear the device during the day with the exception of sleeping, showering, doing strenuous exercise, or working (if the activity resulted in an increase in heart rate). The subject was given instructions on meditation techniques and was asked to replace negative thoughts with happier thoughts or memories of happy occasions or to meditate when alerted of an increase in heart rate over an established threshold. Medications were not used to control depression. Following the use of the device for two weeks, the subject reported that depression episodes were reduced to 9 episodes for the first week and 7 episodes for the second week. For the two week trial period, a total of 16 episodes of depression were reported, representing a reduction in depressive episodes of 36% compared to a two week period before receiving the device. Results are summarized in Table 1.

### Example 3

A subject (CT) suffering from mild to moderate depression was provided with an embodiment of the invention as described in Example 1 and was instructed to wear the device during the day with the exception of sleeping, showering, doing strenuous exercise, or working (if the activity resulted in an increase in heart rate). The subject was given instructions on meditation techniques and was asked to replace negative thoughts with happier thoughts or memories of happy occasions or to meditate when alerted of an increase in heart rate over an established threshold. Medications were not used to control depression. Prior to receiving the device, the subject reported 4 depressive episodes over a 2 week period. During the 2 week trial period, the subject reported a total of 1 episode of depression, representing a reduction in depressive episodes of 75% compared to a two week period before receiving the device. Results are summarized in Table 1.

### Example 4

A subject (P) suffering from regular anxiety attacks, twice requiring hospitalization, was provided with an embodiment of the invention as described in Example 1 and was instructed to wear the device during the day with the exception of sleeping, showering, doing strenuous exercise, or working (if the activity resulted in an increase in heart rate). The subject was given instructions on meditation techniques and was asked to replace negative thoughts with happier thoughts or memories of happy occasions or to meditate when alerted of an increase in heart rate over an established threshold. Medications were not used to control anxiety. Prior to receiving the device, the subject reported having over 30 anxiety attacks per week. During the 2 week trial period, the subject reported 10 anxiety attacks in the first week and 6 anxiety attacks the second week, representing an improvement of 47%. Results are summarized in Table 1.

### Example 5

A subject (MM) suffering from regular anxiety attacks was provided with an embodiment of the invention as described in Example 1 and was instructed to wear the device during the day with the exception of sleeping, showering, doing strenuous exercise, or working (if the activity resulted in an increase in heart rate). The subject was given instructions on meditation techniques and was asked to replace negative thoughts with happier thoughts or memories of happy occasions or to meditate when alerted of an increase in heart rate over an established threshold. Medications were not used to control anxiety. Prior to receiving the device, the subject reported having over 15 anxiety attacks in a 2 week period. During the 2 week trial period, the subject reported 5 anxiety attacks in the first week and 2 anxiety attacks the second week, representing an improvement of 53%. Results are summarized in Table 1.

### Example 6

A subject (FD) suffering from periods of anxiety and panic attacks was provided with an embodiment of the invention as described in Example 1 and was instructed to wear the device during the day with the exception of sleeping, showering, doing strenuous exercise, or working (if the activity resulted in an increase in heart rate). The subject was given instructions on meditation techniques and was asked to replace negative thoughts with happier thoughts or memories of happy occasions or to meditate when alerted of an increase in heart rate over an established threshold. Medications were not used to control anxiety. Prior to receiving the device, the subject reported having at least 5 panic attacks in a 2 week period. During the 2 week trial period, the subject reported having 1 panic attack over the two week trial period, representing an improvement of 80%. Results are summarized in Table 1.

### Example 7

A subject (CZ) suffering from regular periods of anxiety was provided with an embodiment of the invention as described in Example 1 and was instructed to wear the device during the day with the exception of sleeping, showering, doing strenuous exercise, or working (if the activity resulted in an increase in heart rate). The subject was given instructions on meditation techniques and was asked to replace negative thoughts with happier thoughts or memories of happy occasions or to meditate when alerted of an increase in heart rate over an established threshold. Medications were not used to control anxiety. Prior to receiving the device, the subject reported having at least 1 period of anxiety per day. During the 2 week trial period, the subject reported having 3 periods of anxiety in the first week and 2 periods of anxiety in the second week, representing an improvement of 71%. Results are summarized in Table 1.

### Example 8

### FSR Gain and Bias Stage

The recorded signal off of the subject's wrist in rested position was recorded. The base of the input signal was raised by +2 Volts by using V11 and R14 to reach the higher voltage levels for the Frequency Detection stage. This signal goes then through a gain stage. The purpose of this stage is to create an adjustable gain stage through a variable resistor (R22) and an operational amplifier (U11A). An operation amplifier is used instead of a regular voltage divider to reduce current losses due to the high value of the input impedance of an operational amplifier.

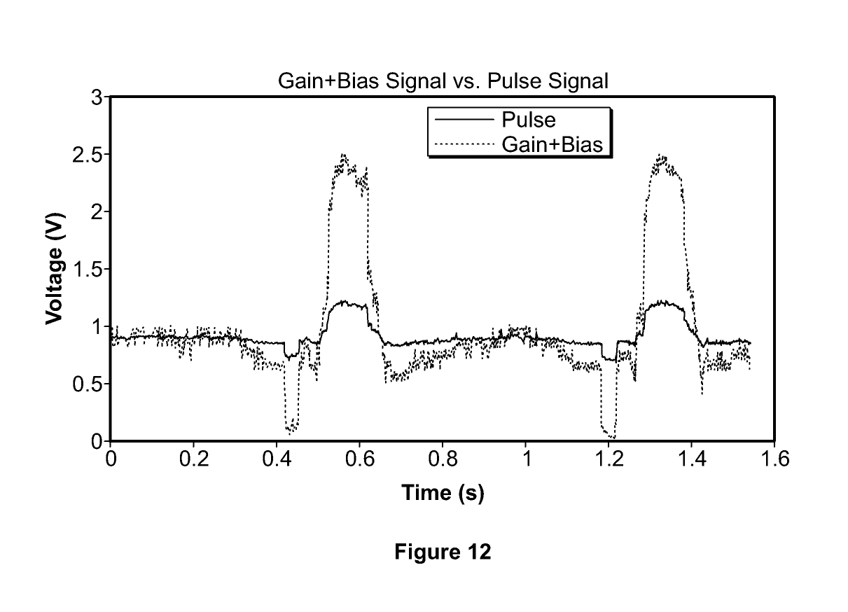

The bias or DC offset of the signal was adjusted by using a second operational amplifier (U11B) and another variable resistor (R25). This latter was polarized positively (V25 of +6 Volts voltage source) from the top and negatively (V26 of −6 Volts voltage source) from the bottom. R23 and R24 resistors limit the current from the voltage sources V25 and V26. When the resistor's position goes toward the positive pole, the signal will be raised positively and when the resistor's position goes toward the negative pole, the signal will be raised negatively. This way the bias or the DC offset of the signal is perfectly controlled and without loss of current since an operational amplifier has been used. R22 and R25 have been adjusted to provide a gain of 3× and a bias adjustment so that both amplified and non-amplified signals have the same voltage level. The input vs. output of this stage is shown in FIG. 12.

### Example 9

### Second Order Low-Pass Filter Stage

A typical 2^{nd} order low-pass filter using an operational amplifier (U2B), two resistors (R11 and R12), and two capacitors (C4 and C5) may be used. Resistors and capacitors are chosen so as to create a filter cut-off frequency of about 20 Hz. The human pulse does not exceed 200 beats per minutes. Therefore the frequency should be smaller than 200 pulse/min/60 s/min=3.33 s⁻¹=3.33 Hz. Therefore a 20 Hz cut-off frequency is well-suited for this application. This filter acts to smooth out all recording noise and mechanical artifacts. It also introduces a slight shift, which is typical for all common filters and does not affect the results in this application. The input vs. output of Low-Pass Filter Stage is shown in FIG. 13.

### Example 10

### Frequency Detector Stage

The Frequency Detector Stage takes the filtered signal, which is the output of the Second Order Low-Pass Filter Stage and compares it to the same signal passed through a diode (D1) and a capacitor (C3). Every time that the signal increases, the capacitor starts being charged. The presence of the diode stops the capacitor to get discharged once charged. A peak detector is created since the capacitor gets charged every time that the input signal increases. The output of the capacitor is then compared to the output of the filter through an operational amplifier (U2A). Every time that the filtered signal goes above the charged signal, U2A creates a high-level signal on its output. As soon as the filtered signal goes below the level of the charged signal, then U2A creates low-level signal on its output. The frequency detector thus converts voltage peaks to a digital pulse. The output of this block can be connected to an external LED that will turn on every time that there is a pulse. By visualizing the pulse via the LED the user can ensure that the device is positioned correctly on the wrist by just checking that the LED beats at the frequency of the person's pulse.

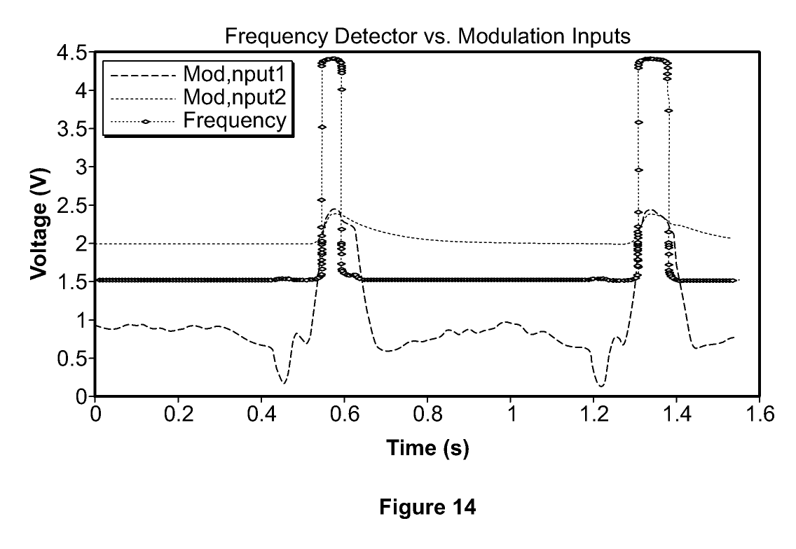

The two inputs vs. output of this block are shown in FIG. 14 for one cycle.

### Example 11

### Frequency-to-Voltage Converter Stage

First the output of the frequency-to-voltage converter is inverted to create a signal that is between 0 Volts and a positive voltage level. This signal is then fed into a LM2907 (U7) chip which is a combination of frequency comparator, frequency-to-voltage comparator, and driver to turn on a buzzer, bulb or similar load. The frequency of the input signal is compared to a threshold frequency and if exceeded, U7 will drive the connected load and activate it. The threshold frequency is set by the following formula: threshold frequency=1/(2*R28*C11)=0.7092 Hz=42.55 beats per minutes. The recorded signal has a frequency of approximately 80 beats per minutes. Therefore the chip activates the buzzer connected to it. The way U7 does that is to change the voltage of its pin8 from +6 Volts to 0 Volts, which in turn will induce a current circulating in the buzzer (U12). When the voltage of pin8 is equal to +6 Volts, both sides of U12 have the same voltage and no current can circulate (device does not buzz). This device needs a second capacitor (C10) to function, which is a charging capacitor that takes few cycles to get charged before U7 functions properly. That is why the output does not change for few cycles before starting to work.

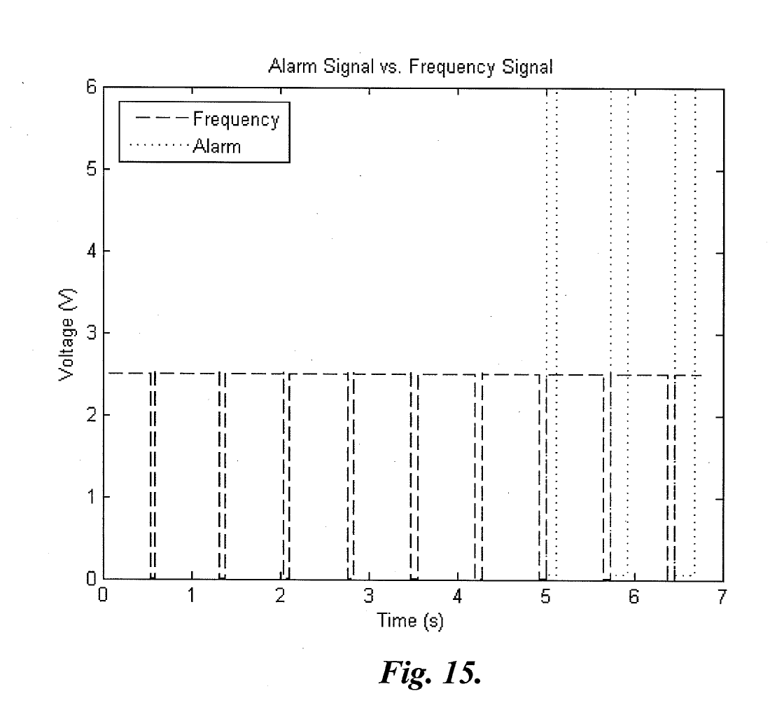

The input vs. output of this block is shown in FIG. 15.

### Example 12

### Vibrator/Alarm Stage

A small linear vibrator is activated by the internal driver of U7. The output signal from U7 is shown in FIG. 15. This signal serves to drive the vibrator (or alternatively buzzer) off and on if the threshold frequency is surpassed.

TABLE 1
Summary of Results of Clinical Study

| | | # Of the attacks per | # of the attacks | Percentage |
|---|---|---|---|---|
| | | two weeks without | per two weeks | of |
| Ref# | Symptom | Pulsar | with Pulsar | Improvement |
| PM | Depression | 25 | 16 | 36 |
| CT | Depression | 4 | 1 | 75 |
| P | Anxiety | 30> | 16 | 46.7 |
| MM | Anxiety | 15 | 7 | 53 |
| FD | Anxiety | 5 | 1 | 80 |
| CZ | Anxiety | 14 | 4 | 71 |

What is claimed is:

1. A method for promoting self-regulation of heart rate, comprising:
a) providing a subject with a compact, wearable biofeedback interface device comprising a pressure sensor which measures the subject's heart rate by converting mechanical force generated by the subject's pulse to a voltage signal, wherein an electronic circuit conditions said signal, processes said signal, compares said processed signal to a pre-selected threshold and when said signal exceeds the threshold, activates a biofeedback stimulus that can be perceived by the subject;
b) instructing the subject to respond to the biofeedback stimulus by practicing a relaxation and/or meditative technique until the subject's heart rate drops below the threshold.

2. The method of claim 1, wherein the subject suffers from an anxiety disorder.

3. The method of claim 2, wherein the anxiety disorder is panic disorder.

4. The method of claim 1, wherein the subject has problems with anger-management.

5. The method of claim 1, wherein the subject suffers from depression.

6. The method of claim 1, wherein the subject's pulse is measured at the radial artery.

7. A method of preventing or reducing the frequency of episodes of anxiety, comprising:
a) wearing a compact wrist-mounted biofeedback device comprising an adjustable wristband provided on the wrist-adjacent surface with a force sensing resistor that changes in input voltage proportional to force applied by pulsations of a radial artery connected to an electrical circuit comprised of a gain and bias stage, a low-pass filter stage, a modulation stage, an LED, a frequency to voltage converter linked to an alarm;
b) receiving a signal from the biofeedback device when measured heart rate exceeds a predetermined threshold;
c) responding to said signal by engaging in relaxation and/or meditative techniques.

8. The method of claim 7, wherein the alarm is a linear vibrator.

9. The method of claim 7, wherein the threshold is set by a variable resistor.

10. A method of preventing or reducing the frequency of episodes of depression, comprising:
a) wearing a compact wrist-mounted biofeedback device comprising an adjustable wristband provided on the wrist-adjacent surface with a force sensing resistor that changes in input voltage proportional to force applied by pulsations of a radial artery connected to an electrical circuit comprised of a gain and bias stage, a low-pass filter stage, a modulation stage, an LED, a frequency to voltage converter linked to an alarm;
b) receiving a signal from the biofeedback device when measured heart rate exceeds a predetermined threshold;
c) responding to said signal by engaging in relaxation and/or meditative techniques.

11. The method of claim 10, wherein the alarm is a linear vibrator.

12. The method of claim 10, wherein the threshold is set by a variable resistor.

13. A kit for promoting self-regulation of heart rate in a subject, comprising:
a) a compact, wearable biofeedback interface device comprising a pressure sensor which measures the subject's heart rate by converting mechanical force generated by the subject's pulse to a voltage signal, wherein an electronic circuit compares said signal to a pre-selected threshold and when said signal exceeds the threshold, activates a biofeedback stimulus that can be perceived by the subject;
b) instructions that teach the subject to respond to the biofeedback stimulus by practicing a relaxation and/or meditative technique until the subject's heart rate drops below the threshold; and
c) an aid to relaxation and/or meditation.

14. The kit of claim 13, wherein the aid is an instructional CD or DVD that teaches the relaxation and/or meditative technique.

15. The kit of claim 13, wherein the aid is a memory device that provides relaxing music and/or video.
